(19) United States
(12) Patent Application Publication
(10) Pub. No.: US 2024/0071627 A1
(43) Pub. Date: Feb. 29, 2024

(54) SYSTEM AND METHOD FOR STRATIFYING AND MANAGING HEALTH STATUS

(71) Applicant: 1Life Healthcare, Inc., San Francisco, CA (US)

(72) Inventor: Rajneesh Behal, San Francisco, CA (US)

(21) Appl. No.: 17/903,884

(22) Filed: Sep. 6, 2022

Related U.S. Application Data
(60) Provisional application No. 63/401,991, filed on Aug. 29, 2022.

(51) Int. Cl. G16H 50/70; G16H 50/30

(52) U.S. Cl. CPC G16H 50/70; G16H 50/30

(57) ABSTRACT

Methods, systems, and non-transitory computer-readable media are configured to perform operations comprising receiving a set of biomarker values associated with a set of individuals; applying a machine learning model to the set of biomarker values to cluster the set of individuals based on the set of biomarker values; segmenting the set of individuals into a selected number of clusters based on the machine learning model; and determining a respective medical classification for each cluster of the selected number of clusters.

### CROSS-REFERENCE TO RELATED APPLICATIONS

This application claims priority to U.S. Provisional Patent Application No. 63/401,991, filed on Aug. 29, 2022 and entitled “SYSTEM AND METHOD FOR STRATIFYING AND MANAGING HEALTH STATUS,” which is incorporated herein by reference in its entirety.

### FIELD OF THE INVENTION

One or more embodiments of the present technology relate to augmenting clinical decision making. More particularly, one or more embodiments of the present technology relate to machine learning techniques for generating medical classifications to target individuals for medical screenings and interventions.

### BACKGROUND

Cardiometabolic diseases are a group of common but often preventable conditions. Cardiometabolic diseases can include, for example, heart disease, stroke, diabetes, insulin resistance, abdominal obesity, non-alcoholic fatty liver disease, hyperglycemia, dyslipidemia, and hypertension. Globally, the number of people who suffer from and are at risk for cardiometabolic diseases continues to increase.

### SUMMARY

Various embodiments of the present technology can include methods, systems, and computer readable media configured to perform operations comprising receiving a set of biomarker values associated with a set of individuals; applying a machine learning model to the set of biomarker values to cluster the set of individuals based on the set of biomarker values; segmenting the set of individuals into a selected number of clusters based on the machine learning model; and determining a respective medical classification for each cluster of the selected number of clusters.

In some embodiments, the machine learning model is an unsupervised machine learning model.

In some embodiments, the set of biomarker values are associated with biomarkers that are readily available.

In some embodiments, the biomarkers include at least one of age, BMI, blood pressure, LDL, HDL, or A1C.

In some embodiments, the selected number of clusters is based on medical knowledge to position a cut on a dendrogram associated with the set of individuals.

In some embodiments, the respective medical classification for each cluster of the selected number of clusters is associated with a level of medical risk for one or more health conditions for individuals associated with the cluster.

In some embodiments, the one or more health conditions are associated with cardiometabolic health conditions.

In some embodiments, the operations further comprise associating a cluster of the selected number of clusters with a level of medical risk for a first health condition; identifying in the cluster a range of biomarker values associated with at least one biomarker that was not known to be indicative of the first health condition; and determining that the range of biomarker values associated with the at least one biomarker is indicative of the first health condition.

In some embodiments, a cluster of the selected number of clusters comprises a subcluster associated with a first level of medical risk for a first health condition that is different from a second level of medical risk for one or more health conditions associated with the cluster.

In some embodiments, the operations further comprise for each cluster of the selected number of clusters, causing a determination of at least one respective action to be performed for individuals associated with the cluster, the at least one respective action including a medical screening or a medical intervention.

It should be appreciated that many other embodiments, features, applications, and variations of the present technology will be apparent from the following detailed description and from the accompanying drawings. Additional and alternative implementations of the methods, systems, and non-transitory computer readable media, and structures described herein can be employed without departing from the principles of the present technology.

### BRIEF DESCRIPTION OF THE DRAWINGS

FIGS. 1A-1C illustrate example simplified functional block diagrams of a multi-entity risk management aid (MERMAID) system, according to various embodiments of the present technology.

FIG. 2 illustrates an example dendrogram of a population of individuals, according to various embodiments of the present technology.

FIG. 3 illustrates an example clustering of a population of individuals, according to various embodiments of the present technology.

FIG. 4 illustrates an example scatterplot matrix of a population of individuals based on their biomarkers, according to various embodiments of the present technology.

FIG. 5 illustrates an example table of respective risk level for cardiometabolic conditions in relation to the corresponding cluster, according to various embodiments of the present technology.

FIG. 6 illustrates an example segmentation of a population into a clustering, according to various embodiments of the present technology.

FIG. 7 illustrates an example diagram of a clustering with associated care strategies, according to various embodiments of the present technology.

FIG. 8 illustrates an example method, according to various embodiments of the present technology.

FIG. 9 illustrates an example computing system to implement one or more embodiments described herein, according to various embodiments of the present technology.

The figures depict various embodiments of the present technology for purposes of illustration only, wherein the figures use like reference numerals to identify like elements. One skilled in the art will readily recognize from the following discussion that alternative embodiments of the methods, systems, and computer readable media illustrated in the figures can be employed without departing from the principles of the present technology described herein.

### DETAILED DESCRIPTION

Cardiometabolic conditions, such as heart disease, stroke, diabetes, insulin resistance, abdominal obesity, non-alcoholic fatty liver disease, hyperglycemia, dyslipidemia, and hypertension, are growing in prevalence. The proliferation of cardiometabolic conditions across all facets of society, such as gender classifications, racial and ethnic groups, and geographic boundaries, is well documented. Currently, cardiometabolic conditions remain the leading causes of death in many populations. However, despite their ubiquity, cardiometabolic conditions are preventable when they are detected early and appropriately managed.

Opportunities to detect and manage cardiometabolic conditions are often tied to clinical care of patients. In general, a visit by a patient to see a health care provider, such as a physician, can include medical observation and routine testing. Such testing can involve panels or individual laboratory tests (or labs) to measure various medical attributes or health traits of the patient. To understand the health of the patient, a physician may analyze a result of a laboratory test performed on the patient to determine a level of risk for a relevant medical condition. When multiple laboratory tests are ordered for a patient, a physician typically will assess the results for each laboratory test in isolation. For example, the physician may determine a level of risk for the patient based on the particular result of a first laboratory test. Likewise, the physician may determine a level of risk for the patient based on the particular result of a second laboratory test. In such circumstances, a more comprehensive level of risk for the patient may elude determination because the physician did not account for all results of multiple laboratory tests together.

For instance, a physician may order an array of laboratory tests for a patient. If the result of one laboratory test in the array indicates that the measured value of LDL of a patient is, for example, 131 or 140, the result can be classified as abnormal because the measured value is greater than 99. Under conventional techniques, a physician may determine a level of risk based on that result alone and accordingly suggest certain treatment or therapy. Because the measured value of LDL is relatively modest, albeit still “abnormal”, the physician may accordingly advise, for example, a more healthful diet and exercise regimen for the patient to prevent heart disease. However, such advice focused solely on the measured value of LDL without regard to results of the other laboratory tests in the array may be incomplete or misleading. In this regard, the measured value of LDL, if combined with results of the other laboratory tests, could potentially warrant determination of a different (e.g., higher) level of risk for heart disease or a level of risk for cardiometabolic conditions beyond merely heart disease. Accordingly, in this example, the measured value of LDL, when assessed in isolation, can result in a misleading or incorrect risk determination.

To supplement their clinical experience and personal medical knowledge, physicians can assess risk for cardiometabolic conditions through use of conventional risk calculators. Conventional risk calculators can be found online and are frequently accessed by health care providers. Physicians can rely on conventional risk calculators as objective, authoritative tools to obtain accurate estimations of risk. Typically, each risk calculator can determine patient risk for a sole cardiometabolic condition. For example, a physician can collect certain patient data—e.g., systolic blood pressure, diastolic blood pressure, total cholesterol, HDL cholesterol, LDL cholesterol—and enter that data into an online calculator based on pooled cohort equations (PCE) provided by the American College of Cardiology (ACC) to estimate the ten-year risk for atherosclerotic cardiovascular disease (ASCVD). As another example, based on age, AST, platelet count, and ALT of a patient, a physician can access a FIB-4 score calculator to generate an estimate of fibrosis in the liver for the patient. As yet other examples, a physician can enter the age, height, weight, and other family and demographic information of a patient into online tests administered by the U.S. Centers for Disease Control and the American Diabetes Association to estimate the risk for, respectively, prediabetes and diabetes. While these and similar risk calculators are useful, each risk calculator is limited to estimating risk for only the particular disease to which it is directed.

Despite the availability of these risk calculators or other testing tools, a physician often does not optimally determine patient risk for multiple cardiometabolic conditions. In many instances, a physician may utilize one risk calculator or other testing tool to acquire an indication of risk for only a particular cardiometabolic condition without further utilization of other risk calculators or testing tools to appropriately determine risks for other potentially relevant cardiometabolic conditions. This may be due to the rigors of modern day medical practice, increasing time constraints on health care providers, cognitive challenge in identifying all potentially relevant cardiometabolic conditions, or some combination of these or other factors. When the physician does not consider risk beyond a sole cardiometabolic condition, patient risk for other cardiometabolic conditions remains unknown. Because cardiometabolic conditions often occur or develop together, medical attention on only one condition can have significant impacts on patient health.

Even where a physician may seek risk determinations for multiple cardiometabolic conditions, conventional techniques present significant disadvantages. For instance, a physician may over test. A physician may indiscriminately order an array of tests, a portion or all of which may be medically unnecessary. For example, a physician may order a stress test for all patients, despite significant differences in the health profiles of the patients. As another example, a physician may order an ultrasound of the liver for all patients without regard to individual health circumstances. Such clinical tendencies subject patients to undue medical testing and related burden, and in the long run are not practically or economically sustainable.

In efforts to determine patient risk for cardiometabolic conditions, conventional techniques also have involved attempts to employ computer technologies. For instance, supervised machine learning techniques have been proposed for risk determinations relating to cardiometabolic conditions. In general, supervised machine learning is a machine learning approach characterized by its use of labeled datasets. The labeled datasets are designed to train algorithms so that they can accurately classify data or predict outcomes. However, use of supervised machine learning techniques for risk determinations relating to cardiometabolic conditions can suffer from significant disadvantages. For example, training data, especially the required labels that reflect outcomes of patients suffering from cardiometabolic diseases, can be challenging to obtain. Acquisition of patient outcome data can be expensive in terms of time and cost. Perhaps even more fundamentally, the availability and amount of such patient outcome data, even if it can be potentially acquired, are often insufficient to adequately train a supervised machine learning model.

An improved approach rooted in technology overcomes the foregoing and other disadvantages associated with conventional approaches specifically arising in the realm of technology. The present technology in various embodiments can enable determination of risk levels for multiple cardiometabolic conditions in a single iteration. The present technology can leverage readily available biomarkers of a patient population of individuals without the need for other types of biomarkers (or proteomics) that would be obtainable only through nonroutine or potentially more cost intensive medical testing. A pre-processing technique can identify individuals associated with records reflecting one or more biomarker values (e.g., readily available biomarker values) that are outliers. Individuals and their biomarker outlier values identified through the pre-processing technique can be eliminated from further consideration. After removal of the individuals associated with biomarker outlier values, the readily available biomarkers of the remaining individuals in the population can be applied to an unsupervised machine learning model. Through a clustering algorithm, the unsupervised machine learning model can generate a clustering that segments the population into a selected number of clusters. The selected number of clusters is configurable, and can be determined based on the clustering algorithm as well as the application of relevant medical knowledge. To validate the clustering, pairs of biomarkers can be separately graphed as scatterplots to confirm that their values vary as expected. Based on the readily available biomarker values, related health data, and medical knowledge, each of the clusters can be assigned a medical classification that describes the cluster. The medical classification can be a level of risk for individuals in the cluster developing cardiometabolic conditions. The risk level for each cluster represents an overall, general estimate of risk (or risk profile) for individuals in the cluster. The risk levels associated with the clusters can constitute categorical priors (or Bayesian priors) to augment clinical decision making by clinicians to prevent or treat cardiometabolic conditions. Determination of a risk level associated with a cluster allows a clinician to better develop an optimal care strategy to combat cardiometabolic conditions for individuals in the cluster.

The present technology overcomes disadvantages associated with conventional techniques. For example, the use of readily available biomarkers in accordance with the present technology obviates the need for expensive data collection efforts and streamlines the dataset for training a machine learning process. As another example, through clustering based on a set of readily available biomarkers—instead of separate consideration of one biomarker after another—the present technology can in one pass determine a more comprehensive assessment of risk for various cardiometabolic conditions. A care strategy for a patient that is generated based on the comprehensive assessment of risk in accordance with the present technology is more effective than a conventional medical plan created from separate assessments of individual health indicators considered in isolation. These and other inventive features and related advantages of the various embodiments of the present technology are discussed in more detail below.

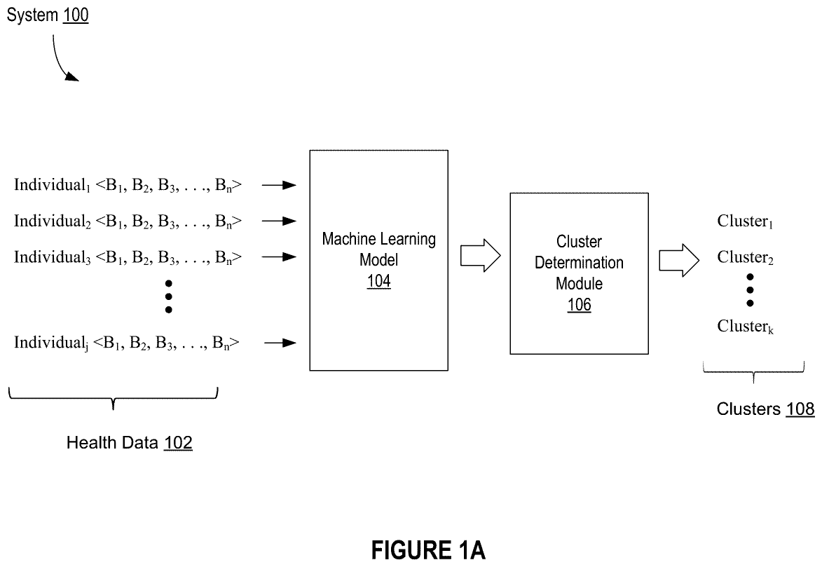

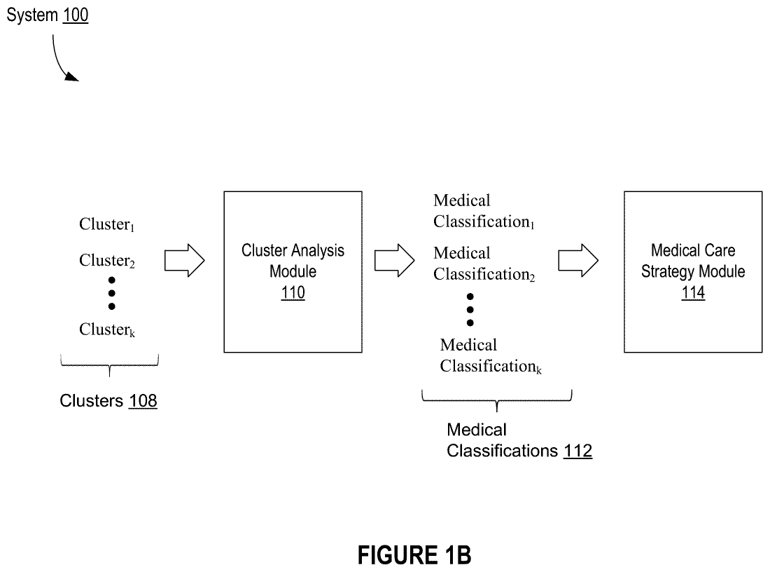

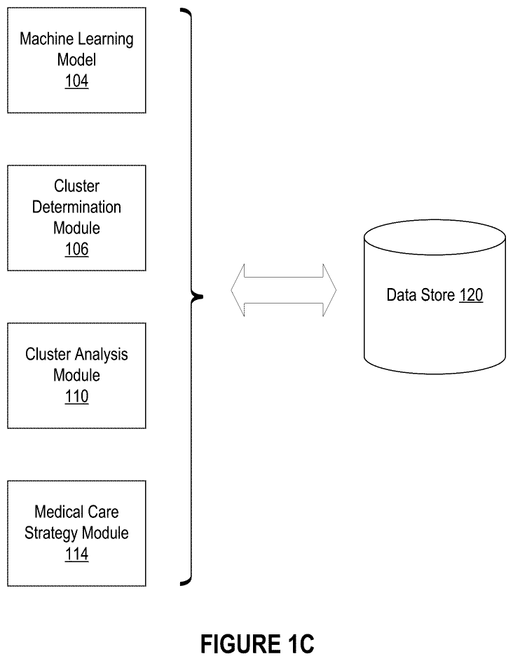

FIGS. 1A-1C illustrate an example multi-entity risk management aid (MERMAID) system 100 to support and augment clinical decision making based on determined risk levels for medical conditions, according to various embodiments of the present technology. The system 100 can generate a clustering having clusters that represent segmentation of a population of individuals based on health data, such as certain biomarkers, of the population. The system 100 can analyze the clusters to determine an associated medical classification for each cluster. For example, the medical classification for each cluster can correspond to a risk profile or level of risk that individuals in the respective cluster will develop medical conditions, such as cardiometabolic conditions, within a time period (e.g., ten years). Based on the risk level for each cluster, the system 100 can support clinical decision making for individuals in the cluster through a health care strategy or plan that is tailored to the cluster and accounts for multiple cardiometabolic conditions. The system 100 thus can allow a clinician to deliver a more informed, comprehensive assessment of risk of a patient developing one or more cardiometabolic conditions based on the cluster into which the patient falls. The determination of cluster membership and a related care strategy for an individual can be advantageously generated in accordance with the present technology with little or no manual action or cognitive effort by a clinician.

In some embodiments, the system 100 can include a machine learning model 104, a cluster determination module 106, a cluster analysis module 110, and a medical care strategy module 114, which are discussed in more detail herein. In some embodiments, some or all of the functionality performed by the system 100 may be performed by one or more computing systems. In some embodiments, some or all of the functionality performed by the system 100 may be performed by one or more backend (or cloud) computing systems or one or more computing systems local to a health care provider office or clinic. In some embodiments, some or all data stored, processed, and utilized by the system 100 can be maintained in one or more data store(s) 120. For example, the data store 120 can include health data of populations, machine learning models to cluster populations, medical knowledge, clustering of populations, medical classifications of clusters, risk levels associated with clusters, care strategies associated with risk levels, etc. The data store 120 can be local or remote to other components (e.g., models, modules, elements, operations, etc.) of the system 100. The components shown in all figures herein, as well as their described functionality, are exemplary only. Other implementations of the present technology may include additional, fewer, integrated, or different components and related functionality. Some components and related functionality may not be shown or described so as not to obscure relevant details. In various embodiments, one or more of the functionalities described in connection with the system 100 can be implemented in any suitable combinations.

The system 100 utilizes health data 102 for a population of individuals. In some embodiments, the population of individuals is a population of persons. The population of persons can be any collection of persons. For example, the population of persons can be some or all members in a group (or a combination of groups) to whom health care services are provided by an organization (or multiple organizations). As other examples, the population of persons can be a representative sampling of a larger population of persons, persons associated with a selected demographic group, persons associated with one or more organizations, persons associated with a geographic region, persons associated with other common attributes or affiliations, etc., or a combination of the foregoing. In some embodiments, the population of individuals can be non-persons. Non-persons can include any living entities susceptible to medical conditions and diseases, such as animals.

In the system 100, the health data 102 of the population of individuals is obtained. The health data 102 can be or include any types of health or medical information relating to the population. In some embodiments, the health data 102 is or includes a set of biomarkers and related biomarker values of the population. In some embodiments, the set of biomarkers can be readily available biomarkers. As used herein, readily available biomarkers are health data 102 about individuals that can be obtained through routine or inexpensive clinical activity.

In some embodiments, a readily available biomarker can be a biomarker that is obtained through clinical activities that satisfy a configurable threshold relating to routineness. Clinical activities, such as patient evaluation by a general physician or a nurse practitioner, patient evaluation by a medical specialist, a patient visit to a laboratory for testing, and the like, can involve or prompt efforts to measure biomarkers of the patient. Such clinical activities can be associated with quantitative values indicating a level or extent to which the clinical activities are routine. As just one example, if a numerical span of routineness values ranges from 10 to 1 with 10 indicating a highest level of routineness, a scheduled annual patient visit to see a primary care physician of the patient may be associated with a routineness value of 9 while a patient visit to see a cardiac imaging specialist because of unusual chest pain may be associated with a routineness value of 3. Accordingly, a biomarker can be considered to be readily available when the biomarker is prompted by or obtained through a clinical activity that satisfies a threshold routineness value. The threshold routineness value is selectable, and can be configurable to have different values in different applications.

In some embodiments, a biomarker can be considered to be a readily available biomarker when the cost to acquire a measure or value of the biomarker satisfies (e.g., falls below) a threshold cost value. For example, cost can be measured in terms of financial expense to determine a value for a biomarker; an amount of laboratory, computer, or human effort to determine a value for a biomarker; time duration required to determine a value for a biomarker; or other expenditure of resources. The threshold cost value is selectable, and can be configurable to have different values in different applications.

In some embodiments, a biomarker can be considered readily available when an applicable threshold routineness value or an applicable threshold cost value is satisfied. In some embodiments, a biomarker can be considered readily available when both an applicable threshold routineness value and an applicable threshold cost value are satisfied. In some embodiments, a biomarker can be considered to be readily available when a person, authority, or organization identifies or declares the biomarker to be a readily available biomarker.

In some embodiments, the set of biomarkers for clustering in accordance with the present technology contains readily available biomarkers that are or include age, BMI (body mass index), blood pressure, LDL (low density lipoproteins), HDL (high density lipoproteins), and A1C (glycated hemoglobin). In some embodiments, the set of biomarkers can include some or all of the readily available biomarkers of age, BMI, blood pressure, LDL, HDL, and A1C, and further include biomarkers other than age, BMI, blood pressure, LDL, HDL, and A1C. The other biomarkers can be other readily available biomarkers, non-readily available biomarkers, or both. In some embodiments, the set of biomarkers does not include any of the biomarkers of age, BMI, blood pressure, LDL, HDL, and A1C, but rather includes other biomarkers. In some embodiments, a biomarker can be excluded from the set of biomarkers when the biomarker is redundant with or correlated to a threshold degree with one or more biomarkers already in the set of biomarkers.

As shown in FIG. 1A, the health data 102 can include health data obtained for each individual in a population of any number of individuals. The population can be expressed as follows:

Population=<Individual₁,Individual₂, . . . ,Individualⱼ>,

where j can be any positive number. Each individual can be associated with a configurable number of biomarkers, such as readily available biomarkers. Values of the biomarkers associated with an individual can be expressed as follows:

Individual=<B₁,B₂, . . . ,Bₙ>,

where n can be any positive number. In some embodiments, n can have a value of 6 that corresponds to the readily available biomarkers of age, BMI, blood pressure, LDL, HDL, and A1C. In some embodiments, n can have a value other than 6 for a different number of biomarkers, including readily available biomarkers or non-readily available biomarkers.

An organization that implements the system 100 can receive the health data 102 of the population of individuals. In some instances, the organization can collect the health data 102 from the individuals directly. In other instances, the health data 102 can be provided to the organization by another entity that collected the health data 102. The organization can be a provider of health care services (e.g., clinic, medical group, hospital, etc.), an organization supporting a provider of health care services, a university, a medical research facility or nonprofit, a government agency involved in health care provision or research, or any other entity or institution directly or indirectly involved in determination of medical risk for diseases or medical interventions to prevent and treat diseases. The organization utilizes the health data 102 in strict compliance with all applicable privacy laws and regulations as well as choices and preferences of the individuals to whom the health data 102 pertains.

In the system 100, the biomarker values associated with the set of biomarkers can be normalized, standardized, or otherwise scaled. In some embodiments, the biomarker values can be scaled to a value in a range between 0 and 1. In some embodiments, the biomarker values can be scaled to have values within a selected range of values other than between 0 and 1.

In some embodiments, a pre-processing technique can be applied to a set of biomarkers associated with a population before clustering of the population. The pre-processing technique can identify individuals in the population with biomarker values (e.g., blood pressure, body weight, etc.) that are considered outliers. A value of a biomarker can be considered an outlier in relation to a distribution of values of the biomarker in a population. As just one example, a person with a body weight of 500 pounds can be considered a person having an outlier value of body weight based on a distribution of body weight values in a population that includes the person. An outlier detection algorithm can be performed to identify biomarker outlier values. For example, an outlier detection algorithm can perform an isolation forest technique, a tree-based unsupervised learning method. Once identified, biomarker outlier values and associated individuals can be eliminated from further consideration and removed from a population before clustering of the remaining individuals in the population based on their biomarker values. Discarding the biomarker outlier values and associated individuals in this manner can advantageously avoid undue shifting of cluster centers.

The set of biomarkers and their values associated with the population can be provided to a machine learning model 104. In some embodiments, the machine learning model 104 can be an unsupervised machine learning model. The unsupervised machine learning model can implement or perform any suitable type of clustering algorithm. In general, clustering the population of individuals can cause individuals with similar biomarker values to be assigned to the same cluster and individuals with dissimilar biomarkers not to be assigned to the same cluster. In some embodiments, the unsupervised machine learning model can implement a hierarchical clustering algorithm to cluster the population based on biomarker values of the set of biomarkers, such as readily available biomarkers. For example, an agglomerative hierarchical clustering algorithm can be based on Ward's linkage or method. In some embodiments, the unsupervised machine learning model can implement any other suitable type of clustering algorithm (e.g., distribution based clustering, density based clustering, grid based clustering, k-means clustering, etc.). In some embodiments, the unsupervised machine learning model does not implement a k-means clustering algorithm. In some embodiments, the unsupervised machine learning model can be periodically retrained based on updated training datasets. For example, the unsupervised machine learning model can be retrained when a population having biomarkers and related values that constitute a potential training dataset has changed (e.g., increased, decreased) by a threshold number of individuals.

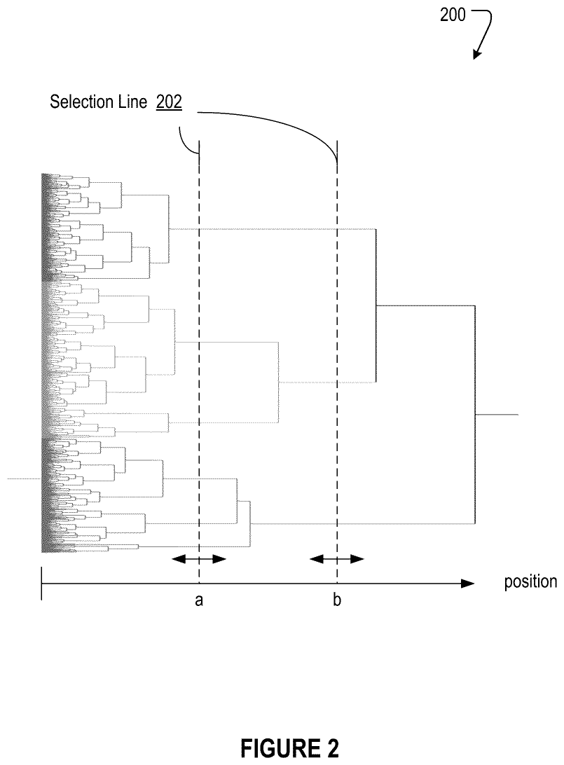

The cluster determination module 106 can evaluate clustering generated by the clustering algorithm implemented by the machine learning model 104. FIG. 2 illustrates a dendrogram 200 of a population of individuals that can be generated and analyzed by the cluster determination module 106, according to various embodiments of the present technology. The dendrogram 200 can graphically represent hierarchical relationships among individuals in the population based on values of their biomarkers, such as readily available biomarkers. Individuals in the population are reflected in the far left of the dendrogram 200. In the dendrogram 200, a relationship between two associated individuals is indicated by a connection, a relationship between pairs of associated individuals is indicated by a connection, and so forth. The dendrogram 200 can facilitate a determination of a selected number of clusters from clustering generated by the clustering algorithm to represent segmentation of the population. The cluster determination module 106 can apply a selection line (or cut) 202 on the dendrogram 200. Based on a position or movement of the selection line 202 along the dendrogram 200, an associated number of clusters can be evaluated to potentially represent the population.

For example, when the cluster determination module 106 positions the selection line 202 to cut through the dendrogram 200 at position a, six horizontal lines are intersected by the selection line 202. The six horizontal lines correspond with six clusters of individuals under consideration to potentially represent the population. The cluster determination module 106 can horizontally move the selection line 202 in either direction to change the number of clusters under consideration. As another example, the cluster determination module 106 can shift the selection line 202 to position b. When the cluster determination module 106 positions the selection line 202 to cut through the dendrogram 200 at position b, three horizontal lines are intersected by the selection line 202. The three horizontal lines correspond with three clusters of individuals under consideration to potentially represent the population of individuals.

Based on the dendrogram 200 and medical knowledge, the cluster determination module 106 can determine a suitable number of clusters in a clustering to represent the population of individuals. In some instances, the cluster determination module 106 can access the data store 120 of the system 100 as shown in FIG. 1C. The data store 120 can contain medical knowledge to support operation and various functionality of the system 100, as described in further detail herein. In some embodiments, medical knowledge can be possessed and utilized by an administrator or user to support operation and function of the system 100, including the cluster determination module 106. Medical knowledge can be based on a vast array of medical information and clinical experience. For example, medical knowledge can include all types of empirical studies, medical research, scientific literature, textbooks, laboratory findings, academic hypotheses, clinical care techniques, clinical results, etc. As other examples, medical knowledge can include information about the structure and function of the body, biomarkers, biomarker correlations, diseases and their indicators, risk levels for disease, diagnostic methodologies, disease prevention, disease management and treatments, and so on.

The data store 120 can contain medical knowledge that the cluster determination module 106 can access and utilize to determine the extent to which two clusters are or should be related. The data store 120 can include medical knowledge that can be used to assess the “clinical distance” between two clusters in determining whether to combine the two clusters into one cluster or to maintain the two clusters as separate. For example, medical knowledge can indicate that, based on biomarker values of individuals in two clusters, the individuals in the two clusters are medically similar to a threshold degree, warranting the two clusters to be combined into one cluster even if the biomarker values between the two clusters are statistically or numerically distinct. In this regard, mere numerical or statistical distinctions in biomarker values between two clusters in some instances may be clinically or medically inconsequential. Thus, in this example, the cluster determination module 106 can combine two clusters into one cluster. As another example, medical knowledge can indicate or suggest that the biomarker values of individuals in two clusters are distinct and indicative of separate medical profiles. In this example, the cluster determination module 106 can maintain the two clusters as separate. The cluster determination module 106 can iteratively and hierarchically analyze pairs (or other groupings) of clusters in this manner until some or all clusters under consideration have been analyzed.

Based on such an analysis, the cluster determination module 106 can determine a clustering of a population that has a suitable or selected number of clusters. A clustering with a selected number of clusters can be expressed as:

Clustering=<Cluster₁,Cluster₂, . . . ,Clusterₖ>,

where k is any selected number of clusters in a clustering. The selected number of clusters is indicated in FIGS. 1A-1B as clusters 108. In some embodiments, the value of k is configurable, and can be different values in different implementations. In some embodiments, a selected number of clusters for representing a population of individuals can be informed or determined by medical practice or convention. Medical risk of individuals for various diseases is often indicated in three risk tiers: low, medium, and high. Accordingly, in some embodiments, clustering of a population of individuals for the purpose of associating medical risk to the individuals can correspond to a selected number of clusters where k is equal to 3. In some embodiments, a different selected number of clusters can be used to represent a different number of tiers of medical risk.

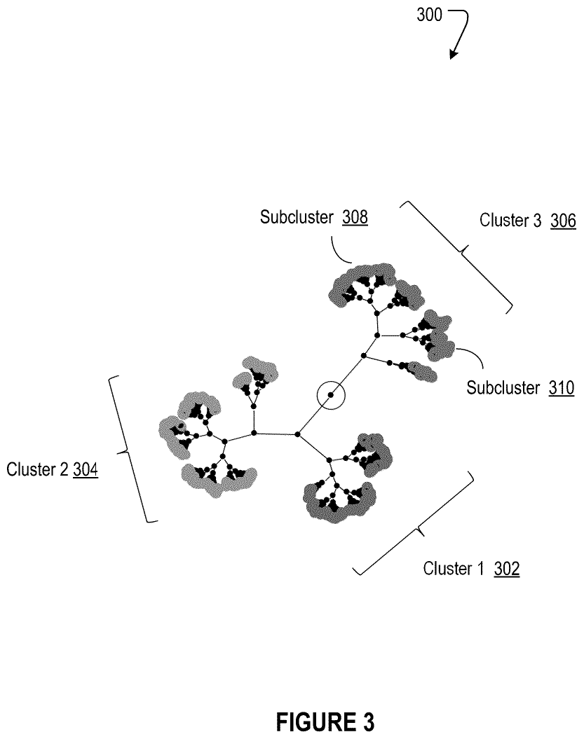

FIG. 3 illustrates an example clustering 300 of a population of individuals, according to various embodiments of the present technology. For example, the clustering 300 can be the clusters 108 of FIGS. 1A-1B. The clustering 300 can reflect a selected number of clusters that appropriately segment the population based on their biomarker values. In some embodiments, the biomarker values can be associated with readily available biomarkers. The selected number of clusters reflected in the clustering 300 can be determined by the cluster determination module 106. In this example, the clustering 300 reflects a selected number of clusters that is equal to 3 (k=3). In other examples, the selected number of clusters can be equal to a value other than 3 (a value less than three, a value more than three). As shown, the clustering 300 includes cluster 1 302, cluster 2 304, and cluster 3 306. As discussed in more detail herein, cluster 1 302, cluster 2 304, and cluster 3 306 can be associated with different medical risk levels for cardiometabolic conditions. In this regard, each cluster of cluster 1 302, cluster 2 304, and cluster 3 306 can be associated with a respective subpopulation that has an associated, unique medical risk level for cardiometabolic conditions. Further, each cluster of cluster 1 302, cluster 2 304, and cluster 3 306 can have its own subclusters, with each subcluster representing a sub-subpopulation. For example, as shown, cluster 3 306 has a multitude of subclusters, including subcluster 308 and subcluster 310, while cluster 1 302 and cluster 2 304 also have their own subclusters. As discussed in more detail herein, each of subcluster 308 and subcluster 310 can represent a respective sub-subpopulation that has an associated medical risk level for cardiometabolic diseases for that sub-subpopulation.

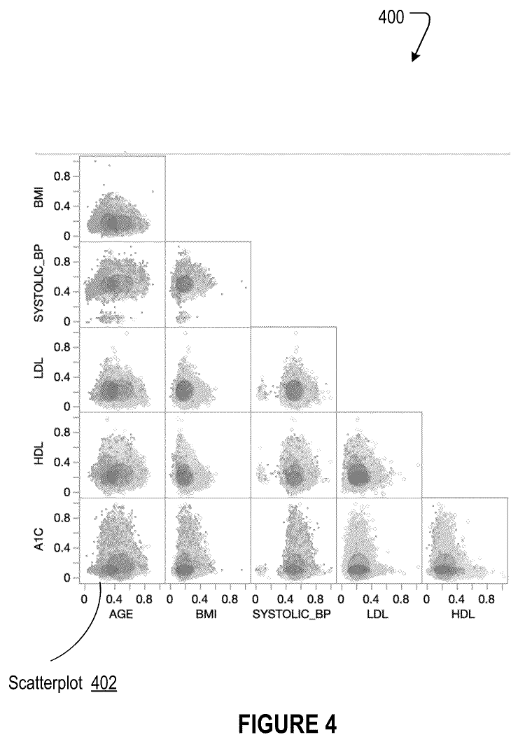

FIG. 4 illustrates an example scatterplot matrix 400 of a population of individuals based on their biomarkers, according to various embodiments of the present technology. In some embodiments, the scatterplot matrix 400 can be generated by the cluster determination module 106. The scatterplot matrix 400 can allow the cluster determination module 106 to validate the clustering 300. Such validation can involve a check and confirmation that observed relationships between biomarkers are sound and consistent with expectations based on medical knowledge. The scatterplot matrix 400 can include a multitude of scatterplots. Each scatterplot in the scatterplot matrix 400 can show a relationship, or absence thereof, between or among a number of biomarkers of the set of biomarkers used to generate the clustering 300. As shown, each scatterplot in the scatterplot matrix 400 shows a relationship between two biomarkers. In other instances, a scatterplot in the scatterplot matrix 400 can show a relationship between any number of biomarkers. A scatterplot of the scatterplot matrix 400 allows confirmation that biomarker values plotted in the scatterplot are correlated (or uncorrelated) as expected. The correctness of the clustering 300 can be verified at least in part when the biomarker values exhibit expected correlation. The scatterplots in the scatterplot matrix 400 can reflect scaled biomarker values, such as values ranging from 0 to 1.

For example, a scatterplot 402 plots values of A1C versus age. The cluster determination module 106 can analyze the relationship between the biomarker values to determine whether they vary in a manner that is consistent with medical knowledge. The scatterplot 402 generally shows relatively low values of A1C in individuals at relatively low values of age. However, as age increases, the scatterplot 402 indicates a range of values of A1C that is relatively higher but consistent. The relationship between the values of A1C and age as reflected in the scatterplot 402 is consistent with medical knowledge. According to medical knowledge, A1C remains at relatively lower values for a younger population because time is required for glucose to accumulate in the body. Thereafter, however, a range of values of A1C can grow in the population, potentially giving rise to prediabetes or diabetes. Accordingly, the scatterplot 402 and the biomarker value correlation reflected therein can be considered to be substantially consistent with medical knowledge. As a result, a confidence level of the clustering 300 can be increased. Correlation, or the absence of correlation, between biomarker values as reflected in other scatterplots of the scatterplot matrix 400 likewise can be analyzed by the cluster determination module 106 and compared to medical knowledge. In some embodiments, the cluster determination module 106 can access medical knowledge in the data store 120 to analyze and validate the clustering 300, as discussed herein. In some embodiments, the cluster determination module 106 can generate the scatterplot matrix 400 for presentation to a clinician so that the clinician can manually validate the clustering 300.

After confirmation of the clustering 300, the clusters 108 of the population of individuals can be provided to the cluster analysis module 110, as shown in FIG. 1B. The cluster analysis module 110 can determine a medical classification (or status, description, indication, profile) for each cluster in the clusters 108. As shown, medical classifications 112 determined by the cluster analysis module 110 can be expressed in general as follows:

Medical Classifications=<Medical Classification₁,Medical Classification₂, . . . ,Medical Classificationₖ>,

where k can be any positive number.

In some embodiments, the medical classification for a cluster can relate to or include a risk profile for individuals in the cluster. The risk profile can relate to a level or extent of medical risk, such as a ten year medical risk or other designation of medical risk for diseases. For example, the level of medical risk can relate to a level of risk for cardiometabolic conditions. To determine risk levels for cardiometabolic conditions associated with the clusters 108, the cluster analysis module 110 can analyze various types of data. For example, the cluster analysis module 110 can analyze biomarkers, such as readily available biomarkers, and their values associated with each cluster. In addition, the cluster analysis module 110 can access and analyze other types of health data about individuals in each cluster that are not readily available biomarkers or their values. Such analysis of the biomarkers and the other types of health data can consider certain numerical ranges of the values of the biomarkers and the other types of health data that can be strong indicators for cardiometabolic conditions. The cluster analysis module 110 also can apply medical knowledge from the data store 120 or possessed by a user of the system 100 to the biomarkers and the other types of health data to determine risk levels for clusters.

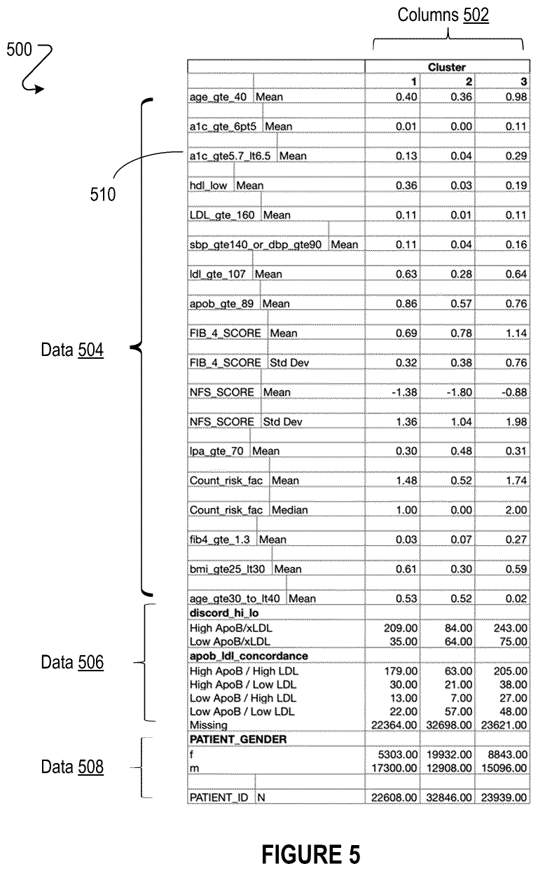

FIG. 5 illustrates an example table 500 through which risk levels for cardiometabolic conditions for the clusters 108 can be determined, according to various embodiments of the present technology. The table 500 can be generated and analyzed by the cluster analysis module 110. For each cluster, the table 500 can contain information relating to readily available biomarkers as well as other types of health data about individuals in each cluster from which a risk level for the cluster can be determined. In the example of table 500, a selected number of clusters to represent a population is 3. Accordingly, the table 500 includes three columns 502 for the subpopulations represented by cluster 1, cluster 2, and cluster 3. In other examples, a selected number of clusters to represent a population can be a value other than 3, as discussed. The table 500 can include various data that can be analyzed for each cluster to determine an associated risk level for cardiometabolic conditions in relation to the cluster. As shown, data 504 of the table 500 includes different types of data relating to readily available biomarkers (e.g., age, A1C, HDL, LDL, systolic blood pressure, diastolic blood pressure, BMI) as well as other types of health data (e.g., ApoB values, FIB-4 scores, NFS scores, Lp(a), etc.) relating to individuals in each of the three clusters. The table 500 considers predetermined numerical ranges of values of the biomarkers and the other types of health data that can reliably indicate cardiometabolic conditions. For example, row 510 indicates that the fraction of the subpopulation in each of cluster 1, cluster 2, and cluster 3 that has an A1C value greater than or equal to 5.7 but less than 6.5 is, respectively, 0.13, 0.04, and 0.29. Data 506 includes values relating to concordance and discordance regarding certain biomarkers (e.g., ApoB, LDL). Data 508 includes patient gender data as well as the population size of each cluster. Based on data 504, 506, 508, the cluster analysis module 110 can apply medical knowledge (e.g., from the data store 120) to generate a risk level for each cluster in the clusters 108. The number of risk levels generated by the cluster analysis module 110 can be equal to the selected number of clusters to represent the population of individuals.

For example, based on data 504, 506, 508 in relation to cluster 3 and application of medical knowledge, the cluster analysis module 110 can observe or determine that more individuals in the ApoB-LDL discordant groups are present in cluster 3. As a result, based on medical knowledge relating to findings published in authoritative medical literature, the cluster analysis module 110 can determine that these individuals are more likely to have non-zero and higher coronary artery calcium (CAC) scores in midlife. The cluster analysis module 110 also can observe that more individuals with higher FIB-4 scores and NFS scores are present in cluster 3 as compared to cluster 1 and cluster 2. In addition, the cluster analysis module 110 can determine that the mean values of these scores for individuals in cluster 3 exceed risk thresholds associated with liver fibrosis and risk of death from liver-related illness or congenital heart disease. The cluster analysis module 110 also can observe that cluster 3 is composed almost exclusively of individuals of age 40 and older, while this age group represents less than half of cluster 1 and cluster 2. The cluster analysis module 110 also can determine that cluster 3 includes a significantly higher proportion of diabetics and prediabetics compared to the other clusters. Accordingly, based on these considerations and characteristics reflective of individuals in cluster 3, the cluster analysis module 110 can associate individuals in cluster 3 with a risk level for cardiometabolic diseases that is highest among the three clusters. Thus, the cluster analysis module 110 can associate cluster 3 with “high” (or relatively higher) risk for cardiometabolic conditions.

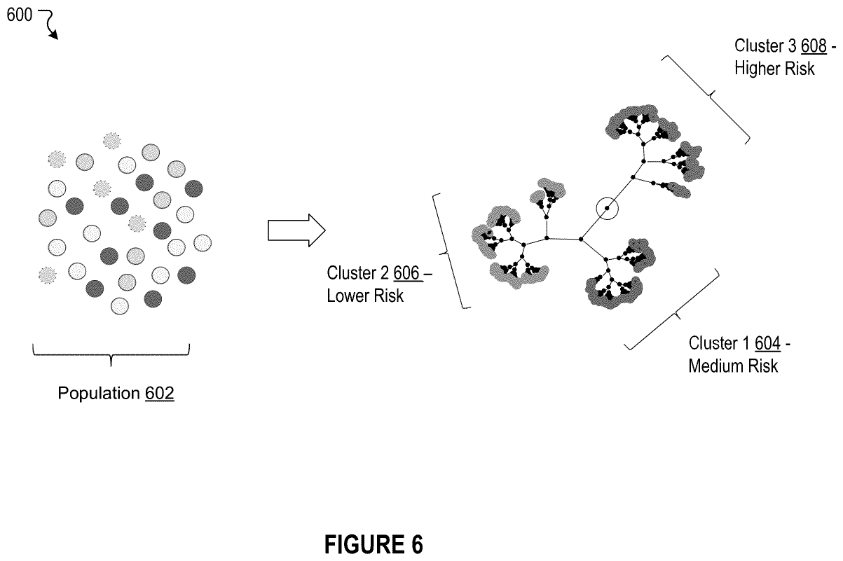

Likewise, the cluster analysis module 110 can determine a risk level for cluster 2 and cluster 1. Based on data 504, 506, 508 in relation to cluster 2 and application of medical knowledge, the cluster analysis module 110 can observe or determine that cluster 2 has a larger number of women; very low rates of diabetes, prediabetes, dyslipidemia, and obesity; and more individuals in cluster 2 with Lp(a) values above 70 in comparison with the other clusters. Accordingly, the cluster analysis module 110 can associate individuals in cluster 2 with a risk level that is lowest among the three clusters. Thus, the cluster analysis module 110 can associate cluster 2 with “low” (or relatively lower) risk for cardiometabolic conditions. The application of medical knowledge to data 504, 506, 508 in relation to cluster 1 in a similar manner can cause the cluster analysis module 110 to associate cluster 1 with a risk level that falls between the risk level associated with cluster 3 and the risk level associated with cluster 2. Accordingly, the cluster analysis module 110 can associate individuals in cluster 1 with “medium” risk for cardiometabolic conditions. FIG. 6 is a diagram 600 illustrating segmentation of a population 602 into a clustering having clusters with associated risk levels for cardiometabolic diseases, according to various embodiments of the present technology. As shown, cluster 1 604, cluster 2 606, and cluster 3 608 are associated with risk levels of, respectively, medium risk, lower risk, and higher risk.

As discussed, each cluster of cluster 1 604, cluster 2 606, and cluster 3 608 can include an array of subclusters. In a manner analogous to determinations of risk level for cardiometabolic conditions for a cluster, each subcluster can be associated with a respective sub-subpopulation that has an associated risk level for cardiometabolic diseases. In a similar manner, each subcluster can be further segmented into sub-subclusters with each sub-subcluster associated with a risk level for cardiometabolic diseases. Accordingly, in general, a cluster, subcluster, sub-subcluster, etc. can be associated with its own risk level for cardiometabolic diseases. In some embodiments, risk levels for all subclusters and sub-subclusters within a cluster are the same. However, in some embodiments, a risk level determined for a subcluster in a cluster can be different than a risk level determined for the cluster. Likewise, a risk level determined for a sub-subcluster in a subcluster can be different than a risk level determined for the subcluster. In this way, the cluster analysis module 110 can provide nuanced determinations of risk levels tailored to a relatively specific group of individuals within a larger group of individuals.

Determinations by the cluster analysis module 110 of risk levels for clusters (or subclusters) can vary. The cluster analysis module 110 can generate different numbers of risk levels to describe clusters of a population. In some embodiments, the number of risk levels can be equal to the number of clusters, with each cluster associated with a respective, unique risk level. In some embodiments, the number of risk levels can be not equal to the number of clusters. For example, the number of risk levels can be less than the number of clusters. In this example, two (or any other number of) clusters may reflect numerically or medically distinct subpopulations while the subpopulations reflect similar or equal levels of risk for cardiometabolic diseases. Thus, the two clusters, while distinct, may be associated with the same risk level. As another example, the number of risk levels can be more than the number of clusters. In this example, the number of risk levels can be the number of clusters plus an additional risk level for a subcluster within a particular cluster that exhibits a risk level that is distinct from its corresponding cluster. The cluster analysis module 110 can generate various types of indications of risk levels. For example, instead of “low” (or “lower”), “medium”, and “high” (or “higher”), the cluster analysis module 110 can generate four risk levels corresponding to “none”, “low”, “medium”, and “high”; or five risk levels corresponding to “none”, “low”, “medium”, “high”, and “highest”; or some other set of designations of risk levels. As another example, the cluster analysis module 110 can assign numerical values to clusters to describe the risk level associated with each cluster. In this example, the cluster analysis module 110 can assign “0” to a cluster associated with relatively lowest risk, “10” to a cluster associated with relatively highest risk, and suitable numbers between 0 and 10 to describe other clusters associated with intermediate risk. Many variations are possible.

The cluster analysis module 110 can discover “new” biomarkers and their values that indicate, in whole or in part, medical risk for cardiometabolic conditions. For example, after determining that a particular cluster of a clustering is associated with, for example, a medium risk or high risk of developing cardiometabolic conditions, the cluster analysis module 110 can analyze biomarkers associated with the cluster. The cluster analysis module 110 may determine that certain biomarkers (e.g., a combination of biomarkers) or their values (e.g., a range of biomarker values) that occur in the cluster do not occur (or do not occur to a threshold extent) in other clusters of the clustering. In this situation, if the biomarkers or their values were not previously known to indicate medium risk or high risk for cardiometabolic conditions, the cluster analysis module 110 can newly identify the biomarkers or their values as indicative of medium risk or high risk for cardiometabolic conditions. With this newfound medical information, the cluster analysis module 110 can add the biomarkers, their values, and their relevance to risk levels for cardiometabolic conditions to the data store 120 of medical knowledge to inform further utilization of the system 100. In some instances, the biomarkers or their values can be subject to further medical research or clinical investigation to confirm their relevance in indicating medical risk for cardiometabolic conditions.

The cluster analysis module 110 can perform feature optimization and dimensionality reduction to improve operation of the machine learning model 104. The cluster analysis module 110 can analyze biomarker values in clusters of a clustering. As just one example, the cluster analysis module 110 can determine that one or more biomarkers and their values occur substantially equally (or similarly to a threshold extent) across all of the clusters. In this situation, the cluster analysis module 110 can infer or determine that the one or more biomarkers and their values are not indicative of a particular risk level for cardiometabolic conditions. As a result, the cluster analysis module 110 can provide to the data store 120 information indicating that the one or more biomarkers and their values are not indicative of risk for cardiometabolic conditions. Accordingly, the cluster analysis module 110 can exclude such biomarkers from datasets to train or retrain the machine learning model 104. The presence of such biomarkers in a dataset to train the machine learning model 104 can constitute noise that negatively impacts training and performance of the machine learning model 104, and can undesirably add to complexity of the machine learning model 104. Thus, the removal of such biomarkers from a training dataset achieves dimensionality reduction to optimize training and performance of the machine learning model 104.

In some embodiments, the cluster analysis module 110 can determine classifications, statuses, descriptions, or other indications of or about clusters representing a population apart from risk levels for cardiometabolic conditions. For example, the cluster analysis module 110 can generate risk levels for clusters in relation to risk for non-cardiometabolic diseases, such as cancer, infectious diseases, skin diseases, neuropsychiatric conditions, etc. The set of biomarkers against which a machine learning model can be applied to generate risk levels for clusters in relation to non-cardiometabolic diseases can include some, all, or none of the biomarkers relevant to determination of risk levels for cardiometabolic diseases. As another example, the cluster analysis module 110 can generate indicators for clusters that do not describe risk levels for diseases. In this example, each indicator associated with a cluster can describe a probability that a medical or health attribute or characteristic other than disease will arise or occur for individuals in the cluster. Many variations are possible.

As shown in FIG. 1B, a medical care strategy module 114 can analyze the medical classifications 112, such as risk levels, for clusters determined by the cluster analysis module 110. Further, the medical care strategy module 114 can analyze information relating to biomarkers (e.g., readily available biomarkers) as well as other types of health data about individuals in each cluster from which a risk level for the cluster can be determined (as discussed in relation to FIG. 5). The medical care strategy module 114 can apply medical knowledge from the data store 120 to the risk levels and the information relating to biomarkers and the other types of health data to generate a care strategy adapted to each cluster and accordingly each individual that falls into the cluster. A care strategy generated by the medical care strategy module 114 can include one or more medical actions to be taken to prevent or treat cardiometabolic conditions. The medical actions can include, for example, medical screenings and medical interventions. In some cases, a care strategy generated by the medical care strategy module 114 can include medical testing, which is not included in routine testing, for medical conditions associated with a corresponding risk level. In addition, the medical care strategy module 114 can determine whether subclusters within a cluster warrant a care strategy that differs from the care strategy for the cluster. Through analysis of health data of individuals in the subcluster, which can include readily available biomarkers as well as other types of health data, the medical care strategy module 114 as warranted can generate a particularized care strategy for the subcluster that is distinct from the general care strategy for the cluster to which the subcluster belongs. A care strategy generated by the medical care strategy module 114 for a cluster (or subcluster) can be provided to a clinician to augment clinical decision making for individuals that fall in the cluster (or subcluster). Such automated generation of the care strategy by the medical care strategy module 114 in accordance with the present technology can ease cognitive demand on the clinician in creation of an appropriate care plan for an individual and can help to avoid mistakes or miscalculations in manual development of the care plan. In some embodiments, operations and functionality of the machine learning model 104, the cluster determination module 106, and the cluster analysis module 110 can be performed as part of a training phase of the present technology, and operations and functionality of the medical care strategy module 114 can be performed as part of a “testing” or operation phase (or evaluation phase) in an application of the present technology. In some embodiments, the medical care strategy module 114 can be implemented by the same organization that implements the machine learning model 104, the cluster determination module 106, and the cluster analysis module 110. In some embodiments, the medical care strategy module 114 can be implemented by an organization that is different or separate from another organization that implements the machine learning model 104, the cluster determination module 106, and the cluster analysis module 110. For example, a first organization can determine the medical classifications 112 associated with the clusters 108, and provide such information to one or more second organizations to allow the second organizations to develop their own, unique care strategies for the medical classifications 112.

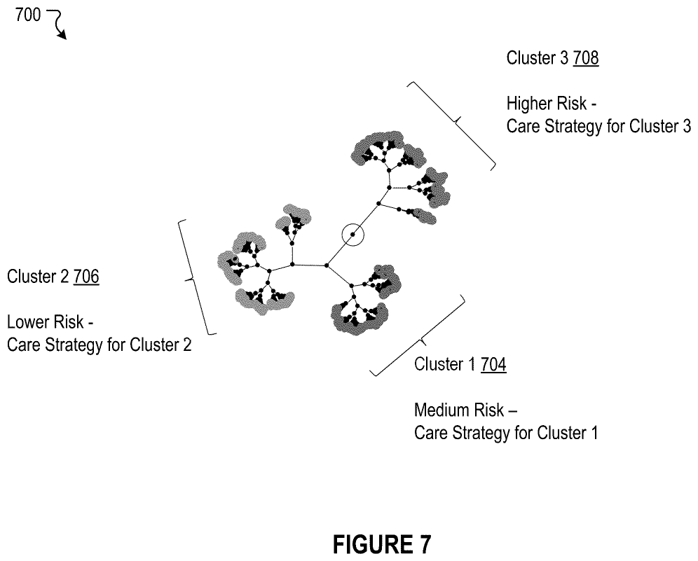

FIG. 7 is a diagram 700 of a clustering with associated care strategies for cluster 1 704, cluster 2 706, and cluster 3 708, which correspond to, respectively, cluster 1 604, cluster 2 606, and cluster 3 608 of FIG. 6. In FIG. 7, cluster 1 704, cluster 2 706, and cluster 3 708 are associated with, respectively, medium risk, lower risk, and higher risk. Based on its corresponding risk level, each of cluster 1 704, cluster 2 706, and cluster 3 708 can be associated with a tailored care strategy for individuals that fall in the cluster. For example, individuals for whom the medical care strategy module 114 can generate care strategies can include individuals associated with biomarkers and their values that were used to train the machine learning model 104, generate the clusters 108, and determine the medical classifications 112. As another example, individuals for whom the medical care strategy module 114 can generate care strategies can include individuals that are assigned to the clusters 108 based on their biomarkers and biomarker values after training of the machine learning model 104, generation of the clusters 108, and determination of the medical classifications 112 for the clusters. In this example, training of the machine learning model 104, generation of the clusters 108, and determination of medical classifications 112 for the clusters can be based on a population of individuals. Thereafter, an individual not included in the population can be assigned to an already generated cluster with an associated medical classification (e.g., risk level for cardiometabolic conditions) based on biomarker values of the individual.

The medical care strategy module 114 can generate a plan (e.g., primary care plan) reflecting an appropriate care strategy for individuals that fall into each cluster. Because cluster 3 708 is associated with higher risk, the medical care strategy module 114 also can recommend enrollment of individuals in cluster 3 708 in a tailored intervention program. The program can be directed at individuals with diabetes, prediabetes, hypertension, or obesity. The program can subject the individuals to intensive lifestyle management and potentially aggressive medications. In addition, because of the higher risk associated with cluster 3 708, the medical care strategy module 114 can calculate 10 year risk or lifetime risk for ASCVD, and selectively order a CT-CAC (coronary artery calcium heart scan) or a Cor-CTA (coronary CT angiography). Further, the medical care strategy module 114 can recommend statins and appropriate lifestyle management for individuals in cluster 3 708. For individuals having FIB-4 scores or NFS scores that satisfy score thresholds, the medical care strategy module 114 can recommend a FibroScan to assess likelihoods for developing liver fibrosis or fatty liver.

With respect to a care strategy for cluster 1 704, the medical care strategy module 114 can recommend enrollment of individuals with diabetes, prediabetes, or obesity in a tailored intervention program that provides intensive lifestyle management and medication management. The medical care strategy module 114 can calculate 10 year risk or lifetime risk for ASCVD, and selectively order a CT-CAC (coronary artery calcium heart scan) or a Cor-CTA (coronary CT angiography). Further, the medical care strategy module 114 can recommend statins and appropriate lifestyle management for individuals in cluster 1 704. In addition, for cluster 1 704, the medical care strategy module 114 can selectively order laboratories of ApoB and Lp(a), especially for younger individuals in the cluster who otherwise may appear to have low risk.

With respect to a care strategy for cluster 2 706, the medical care strategy module 114 can calculate 10 year risk or lifetime risk for ASCVD, and selectively order a CT-CAC (coronary artery calcium heart scan) or a Cor-CTA (coronary CT angiography). Further, the medical care strategy module 114 can recommend statins and appropriate lifestyle modification for individuals in cluster 2 706. In addition, for a specific subcluster in cluster 2 706 associated with women during menopause transition years or post-menopause, or those with early menopause, the medical care strategy module 114 can recommend enrollment of these women in a tailored intervention program with a focus on lifestyle management and additional risk assessment.

In some embodiments, the medical care strategy module 114 can identify a subcluster within the cluster 2 706 that may warrant a care strategy that is distinct from the care strategy for cluster 2 706. For example, cluster 2 706 can include a subcluster that contains a threshold number of individuals with Lp(a) values that are relatively high (or satisfy an Lp(a) threshold value). Because Lp(a) is a direct risk factor in development of cardiovascular disease (e.g., heart attack, stroke), the medical care strategy module 114 can determine that a tailored care strategy for the individuals in the subcluster is warranted. The care strategy for the individuals in the subcluster can include medical screenings and medical interventions that are, for example, additional to medical screenings and medical interventions in the care strategy for individuals in the cluster generally. Accordingly, the medical care strategy module 114 can identify specific health attributes or characteristics of individuals in a subcluster that may call for more focused medical care than otherwise would be provided for individuals in the related cluster. The medical care strategy module 114 may recommend such focused medical care for individuals in a subcluster even when the corresponding cluster is associated with low risk for cardiometabolic conditions.

The care strategies determined by the medical care strategy module 114 as discussed herein are examples. As informed by medical knowledge, the medical care strategy module 114 can develop care strategies and related medical actions to be taken other than those expressly referenced herein. For example, for a given cluster with an associated risk level, the medical care strategy module 114 can generate care strategies that omit one or more of the medical actions discussed herein for the care strategies associated with cluster 1 704, cluster 2 706, and cluster 3 708. As another example, the medical care strategy module 114 can generate care strategies that include one or more additional medical actions not discussed herein in relation to the care strategies associated with the cluster 1 704, cluster 2 706, and cluster 3 708. Understanding in the medical community of cardiometabolic conditions, and related preventive measures and treatments, will continue to increase. As this understanding increases, the care strategies associated with cluster 1 704, cluster 2 706, and cluster 3 708 likewise can change to reflect the increased understanding. The data store 120 can be continuously updated with new medical information as it becomes understood in the medical community to support the generation of care strategies by the medical care strategy module 114.

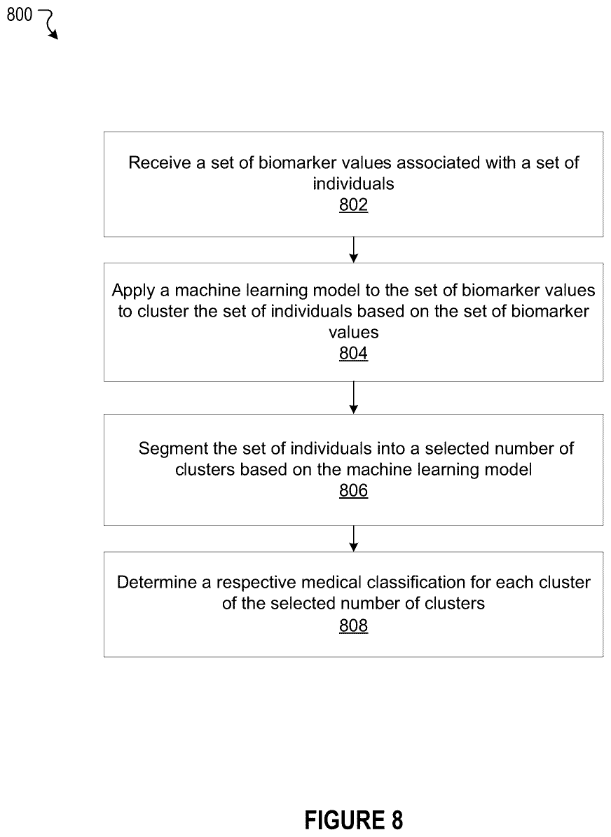

FIG. 8 illustrates an example method 800, according to various embodiments of the present technology. At 802, the method 800 can receive a set of biomarker values associated with a set of individuals. At 804, the method 800 can apply a machine learning model to the set of biomarker values to cluster the set of individuals based on the set of biomarker values. At 806, the method 800 can segment the set of individuals into a selected number of clusters based on the machine learning model. At 808, the method 800 can determine a respective medical classification for each cluster of the selected number of clusters. It should be appreciated that there can be additional, fewer, or alternative steps performed in similar or alternative orders, or in parallel, within the scope of the various embodiments discussed herein unless otherwise stated.

It is contemplated that there can be many other uses, applications, and variations associated with the various embodiments of the present technology. As referenced herein, determinations of risk for cardiometabolic conditions are discussed as examples. However, the present technology also can apply to determinations of risk for various other types of medical conditions and diseases apart from cardiometabolic conditions and diseases. For example, health data, such as readily available health attributes or biomarkers relevant to the other types of medical conditions and diseases, can be used to train a machine learning model to group a relevant population into clusters. Each cluster can be associated with various risk levels associated with the other types of medical conditions and diseases that do not relate to cardiometabolic conditions and diseases. Tailored advice regarding medical screenings and interventions for the other types of medical conditions and diseases can be associated with each cluster to augment clinical decision making. Further, many examples set forth herein discuss automatic (computerized) provision and application of medical knowledge to support operations and functionalities of the present technology. For example, the cluster determination module 106, the cluster analysis module 110, and the medical care strategy module 114 can automatically (electronically) communicate with the data store 120 to access and utilize medical knowledge maintained in the data store 120 to support or carry out their computerized operations and functionalities, as shown in FIG. 1C. However, the present technology can also apply to implementations where medical knowledge instead is possessed by health care providers (e.g., clinicians) and manually provided by the health care providers to the system 100 to support some or all of the operations and functionalities of the present technology. Further still, while a population of individuals is discussed herein with respect to persons in various examples, the present technology also can be applied to a population of any individuals or entities, such as nonhumans. For example, the present technology can be applied to animals. In this example, determinations of risk may be associated with medical conditions and diseases that typically afflict animals. Likewise, clustering of a population of animals may be based on readily available health data or biomarkers associated with the animals. Moreover, risk levels are discussed herein as medical classifications associated with clusters of a population. The present technology, however, can apply to other types of medical classifications other than risk levels. For example, the medical classifications can include any other health attribute or medical characteristic of individuals in the population. Many variations of the present technology are possible.

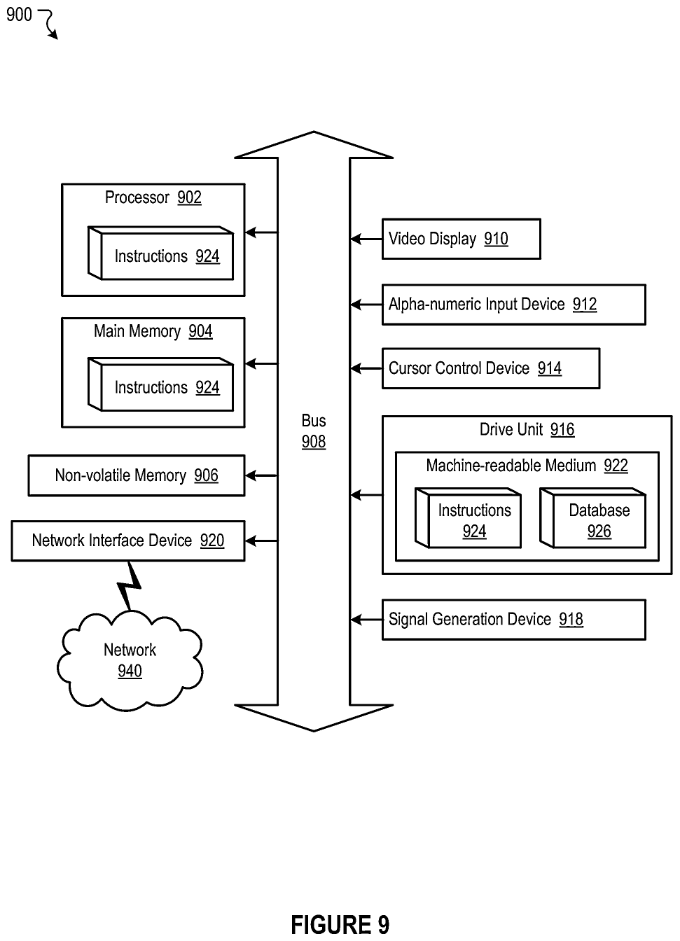

FIG. 9 illustrates an example of a computing system (or computing device) 900 that may be used to implement one or more of the embodiments of the present technology, or components thereof, as described herein according to various embodiments of the present technology. The computer system 900 can be included in a wide variety of local and remote machine and computer system architectures and in a wide variety of network and computing environments that can implement the functionalities of the present technology. The computing system 900 includes sets of instructions 924 for causing the computing system 900 to perform the processes and features discussed herein. For example, the sets of instructions 924 can include instructions configured to carry out or implement operations and functionality described in relation to the machine learning model 104, the cluster determination module 106, the cluster analysis module 110, and the medical care strategy module 114. The computing system 900 may be connected (e.g., networked) to other machines and/or computer systems. In a networked deployment, the computing system 900 may operate in the capacity of a server or a client machine in a client-server network environment, or as a peer machine in a peer-to-peer (or distributed) network environment. The present technology can be implemented by the computing system 900 alone. In addition, the present technology can be implemented across an array of two or more of the computing systems 900 constituting a distributed computing architecture. For example, each computing system 900 of the array can implement a portion of the functionality of the present technology.

The computing system 900 includes a processor 902 (e.g., a central processing unit (CPU), a graphics processing unit (GPU), or both), a main memory 904, and a nonvolatile memory 906 (e.g., volatile RAM and non-volatile RAM, respectively), which communicate with each other via a bus 908. For example, memory of the computing system 900, such as the nonvolatile memory 906, can include the data store 120. In some embodiments, the computing system 900 can be a desktop computer, a laptop computer, personal digital assistant (PDA), or mobile phone, for example. In one embodiment, the computing system 900 also includes a video display 910, an alphanumeric input device 912 (e.g., a keyboard), a cursor control device 914 (e.g., a mouse), a drive unit 916, a signal generation device 918 (e.g., a speaker), and a network interface device 920.

In one embodiment, the video display 910 includes a touch sensitive screen for user input. In one embodiment, the touch sensitive screen is used instead of a keyboard and mouse. The disk drive unit 916 includes a machine-readable medium 922 on which is stored one or more sets of instructions 924 (e.g., software) embodying any one or more of the methodologies or functions described herein. The instructions 924 can also reside, completely or at least partially, within the main memory 904 and/or within the processor 902 during execution thereof by the computing system 900. The instructions 924 can further be transmitted or received over a network 940 via the network interface device 920. In some embodiments, the machine-readable medium 922 also includes a database 926.

Volatile RAM may be implemented as dynamic RAM (DRAM), which requires power continually in order to refresh or maintain the data in the memory. Non-volatile memory is typically a magnetic hard drive, a magnetic optical drive, an optical drive (e.g., a DVD RAM), or other type of memory system that maintains data even after power is removed from the system. The non-volatile memory 906 may also be a random access memory. The non-volatile memory 906 can be a local device coupled directly to the rest of the components in the computing system 900. A non-volatile memory that is remote from the system, such as a network storage device coupled to any of the computer systems described herein through a network interface such as a modem or Ethernet interface, can also be used.

While the machine-readable medium 922 is shown in an exemplary embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present technology. Examples of machine-readable media (or computer-readable media) include, but are not limited to, recordable type media such as volatile and non-volatile memory devices; solid state memories; floppy and other removable disks; hard disk drives; magnetic media; optical disks (e.g., Compact Disk Read-Only Memory (CD ROMS), Digital Versatile Disks (DVDs)); other similar non-transitory (or transitory), tangible (or non-tangible) storage medium; or any type of medium suitable for storing, encoding, or carrying a series of instructions for execution by the computing system 900 to perform any one or more of the processes and features described herein.

In general, routines executed to implement the embodiments of the invention can be implemented as part of an operating system or a specific application, component, program, object, module, or sequence of instructions referred to as “programs” or “applications”. For example, one or more programs or applications can be used to execute any or all of the functionality, techniques, and processes described herein. The programs or applications typically comprise one or more instructions set at various times in various memory and storage devices in the machine and that, when read and executed by one or more processors, cause the computing system 600 to perform operations to execute elements involving the various aspects of the embodiments described herein.

The executable routines and data may be stored in various places, including, for example, ROM, volatile RAM, non-volatile memory, and/or cache memory. Portions of these routines and/or data may be stored in any one of these storage devices. Further, the routines and data can be obtained from centralized servers or peer-to-peer networks. Different portions of the routines and data can be obtained from different centralized servers and/or peer-to-peer networks at different times and in different communication sessions, or in a same communication session. The routines and data can be obtained in entirety prior to the execution of the applications. Alternatively, portions of the routines and data can be obtained dynamically, just in time, when needed for execution. Thus, it is not required that the routines and data be on a machine-readable medium in entirety at a particular instance of time.

While embodiments have been described fully in the context of computing systems, those skilled in the art will appreciate that the various embodiments are capable of being distributed as a program product in a variety of forms, and that the embodiments described herein apply equally regardless of the particular type of machine- or computer-readable media used to actually affect the distribution.

Alternatively, or in combination, the embodiments described herein can be implemented using special purpose circuitry, with or without software instructions, such as using Application-Specific Integrated Circuit (ASIC) or Field-Programmable Gate Array (FPGA). Embodiments can be implemented using hardwired circuitry without software instructions, or in combination with software instructions. Thus, the techniques are limited neither to any specific combination of hardware circuitry and software, nor to any particular source for the instructions executed by the data processing system.

For purposes of explanation, numerous specific details are set forth in order to provide a thorough understanding of the description. It will be apparent, however, to one skilled in the art that embodiments of the technology can be practiced without these specific details. In some instances, modules, structures, processes, features, and devices are shown in block diagram form in order to avoid obscuring the description discussed herein. In other instances, functional block diagrams and flow diagrams are shown to represent data and logic flows. The components of block diagrams and flow diagrams (e.g., modules, engines, blocks, structures, devices, features, etc.) may be variously combined, separated, removed, reordered, and replaced in a manner other than as expressly described and depicted herein.

Reference in this specification to “one embodiment”, “an embodiment”, “other embodiments”, “another embodiment”, “in various embodiments”, “for example”, “for instance”, “in one implementation”, or the like means that a particular feature, design, structure, or characteristic described in connection with the embodiment is included in at least one embodiment of the technology. The appearances of, for example, the phrases “according to an embodiment”, “in one embodiment”, “in an embodiment”, “in some embodiments”, “in various embodiments”, or “in another embodiment” in various places in the specification are not necessarily all referring to the same embodiment, nor are separate or alternative embodiments mutually exclusive of other embodiments. Moreover, whether or not there is express reference to an “embodiment” or the like, various features are described, which may be variously combined and included in some embodiments but also variously omitted in other embodiments. Similarly, various features are described which may be preferences or requirements for some embodiments but not other embodiments.

Although embodiments have been described with reference to specific exemplary embodiments, it will be evident that the various modifications and changes can be made to these embodiments. Accordingly, the specification and drawings are to be regarded in an illustrative sense rather than in a restrictive sense. The foregoing specification provides a description with reference to specific exemplary embodiments. It will be evident that various modifications can be made thereto without departing from the broader spirit and scope as set forth in the following claims. The specification and drawings are, accordingly, to be regarded in an illustrative sense rather than a limiting or restrictive sense.

Although some of the drawings illustrate a number of operations or method steps in a particular order, steps that are not order dependent may be reordered and other steps may be combined or omitted. While some reordering or other groupings are specifically mentioned, others will be apparent to those of ordinary skill in the art and so do not present an exhaustive list of alternatives. Moreover, it should be recognized that the stages could be implemented in hardware, firmware, software, or any combination thereof.

It should also be understood that a variety of changes may be made without departing from the essence of the invention. Such changes are also implicitly included in the description. They still fall within the scope of this invention. It should be understood that this technology is intended to yield a patent covering numerous aspects of the invention, both independently and as an overall system, and in both method and apparatus modes.

Further, each of the various elements of the invention and claims may also be achieved in a variety of manners. This technology should be understood to encompass each such variation, be it a variation of an embodiment of any apparatus embodiment, a method or process embodiment, or even merely a variation of any element of these.

Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising” are intended to imply the inclusion of a stated element or step or group of elements or steps, but not the exclusion of any other element or step or group of elements or steps. Such terms should be interpreted in their most expansive forms so as to afford the applicant the broadest coverage legally permissible in accordance with the following claims.

The language used herein has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the technology of the embodiments of the invention is intended to be illustrative, but not limiting, of the scope of the invention, which is set forth in the following claims.

What is claimed is:

1. A computer-implemented method comprising:
receiving, at a computing system, a set of biomarker values associated with a set of individuals;
applying, by the computing system, a machine learning model to the set of biomarker values to cluster the set of individuals based on the set of biomarker values;
segmenting, by the computing system, the set of individuals into a selected number of clusters based on the machine learning model; and
determining, by the computing system, a respective medical classification for each cluster of the selected number of clusters.

2. The computer-implemented method of claim 1, wherein the machine learning model is an unsupervised machine learning model.

3. The computer-implemented method of claim 1, wherein the set of biomarker values are associated with biomarkers that are readily available.

4. The computer-implemented method of claim 3, wherein the biomarkers include at least one of age, BMI, blood pressure, LDL, HDL, or A1C.

5. The computer-implemented method of claim 1, wherein the selected number of clusters is based on medical knowledge to position a cut on a dendrogram associated with the set of individuals.

6. The computer-implemented method of claim 1, wherein the respective medical classification for each cluster of the selected number of clusters is associated with a level of medical risk for one or more health conditions for individuals associated with the cluster.

7. The computer-implemented method of claim 6, wherein the one or more health conditions are associated with cardiometabolic health conditions.

8. The computer-implemented method of claim 6, further comprising:
associating, by the computing system, a selected cluster of the selected number of clusters with a level of medical risk for a first health condition;
identifying, by the computing system, in the selected cluster a range of biomarker values associated with at least one biomarker that was not known to be indicative of the first health condition; and
determining, by the computing system, that the range of biomarker values associated with the at least one biomarker is indicative of the first health condition.

9. The computer-implemented method of claim 6, wherein a cluster of the selected number of clusters comprises a subcluster associated with a first level of medical risk for a first health condition that is different from a second level of medical risk for one or more health conditions associated with the cluster.

10. The computer-implemented method of claim 1, further comprising:
for each cluster of the selected number of clusters, causing a determination of at least one respective action to be performed for individuals associated with the cluster, the at least one respective action including a medical screening or a medical intervention.

11. A system comprising:
at least one processor; and
a memory storing instructions that, when executed by the at least one processor, cause the system to perform operations comprising:
receiving a set of biomarker values associated with a set of individuals;
applying a machine learning model to the set of biomarker values to cluster the set of individuals based on the set of biomarker values;
segmenting the set of individuals into a selected number of clusters based on the machine learning model; and
determining a respective medical classification for each cluster of the selected number of clusters.

12. The system of claim 11, wherein the machine learning model is an unsupervised machine learning model.

13. The system of claim 11, wherein the set of biomarker values are associated with biomarkers that are readily available.

14. The system of claim 13, wherein the biomarkers include at least one of age, BMI, blood pressure, LDL, HDL, or A1C.

15. The system of claim 11, wherein the selected number of clusters is based on medical knowledge to position a cut on a dendrogram associated with the set of individuals.

16. A non-transitory computer-readable storage medium including instructions that, when executed by at least one processor of a computing system, cause the computing system to perform operations comprising:
receiving a set of biomarker values associated with a set of individuals;
applying a machine learning model to the set of biomarker values to cluster the set of individuals based on the set of biomarker values;
segmenting the set of individuals into a selected number of clusters based on the machine learning model; and
determining a respective medical classification for each cluster of the selected number of clusters.

17. The non-transitory computer-readable storage medium of claim 16, wherein the machine learning model is an unsupervised machine learning model.

18. The non-transitory computer-readable storage medium of claim 16, wherein the set of biomarker values are associated with biomarkers that are readily available.

19. The non-transitory computer-readable storage medium of claim 18, wherein the biomarkers include at least one of age, BMI, blood pressure, LDL, HDL, or A1C.

20. The non-transitory computer-readable storage medium of claim 16, wherein the selected number of clusters is based on medical knowledge to position a cut on a dendrogram associated with the set of individuals.
